(19) United States
(12) Patent Application Publication
(10) Pub. No.: US 2020/0253554 A1
(43) Pub. Date: Aug. 13, 2020

(54) NONINVASIVE SPINAL TRACKING

(71) Applicant: DePuy Synthes Products, Inc., Raynham, MA (US)

(72) Inventors: Steffen Schumann, Zofingen (CH); Marc Puls, Thörigen (CH)

(21) Appl. No.: 16/275,007

(22) Filed: Feb. 13, 2019

(51) Int. Cl. A61B 5/00; A61B 34/20

(52) U.S. Cl. CPC A61B 5/6833; A61B 2034/2055; A61B 5/6823; A61B 34/20

(57) ABSTRACT

Non-invasive tracking methods and devices are provided that are configured to track at least one selectable internal element within a patient, such as a vertebra, relative to an external point outside of the patient's body, such as a reference point within an operating room. For example, in one embodiment, a patch is provided and is configured to be removably attached to skin of a patient, and to track a distance relative to the patch of at least one selectable element within a patient. The patch can also be configured to be tracked by an external monitoring system that tracks its orientation and location relative to the monitoring system, thereby indicating a position of a vertebra.

### FIELD

Surgical devices, systems, and methods are provided for tracking locations of various elements within a patient noninvasively.

### BACKGROUND

During various spinal surgeries, it can be important to a surgeon to be able to track the locations of various spinal elements of a patient and locations of surgical instruments with respect to the spinal elements to limit harm to the patient while more effectively performing the operation. As such, various methods have been developed to assist in tracking spinal elements, such as vertebra. However, these methods are generally invasive and complicated without providing effective navigation in real-world conditions. In one such method, a physical reference device is attached to a single vertebral body of the patient, and the device extends outside of the patient to provide a point of reference for the surgeon when operating on remaining vertebral bodies. Thus, this approach is not only invasive, but it also depends on the various vertebral bodies remaining in a fixed orientation with respect to the reference vertebral body. Any vertebral movement during the operation, such as disc removal, patient shifting, etc., will render the reference tracking useless.

Thus, there remains a need for surgical instruments, methods, and systems for noninvasively tracking various elements within a patient.

### SUMMARY

Accordingly, tracking methods and devices are provided herein that allow non-invasive location tracking of various elements, and especially various spinal bodies, within a patient. In one aspect, a surgical tracking device is provided that has at least one patch. The at least one patch has a patient-facing surface configured to be removably attached to skin of a patient and an outward-facing surface. The patch also has at least one sensor therein that is configured to sense a distance between the at least one sensor and at least one selectable vertebra within the patient. At least one target is positioned on the patch, and the relative orientation and location of the at least one target is configured to be tracked by an external monitoring system.

The device can have numerous variations. For example, the at least one sensor can be in the form of at least one ultrasound sensor. The at least one sensor of the at least one patch can also be configured to track at least one of a total distance from the at least one patch to the at least one vertebra and an axial rotation of the at least one vertebra relative to the at least one patch over time. In one embodiment, the at least one patch can be a stretchable ultrasound patch, and the at least one sensor can include an array of piezoelectronic ultrasonic transducers. In other aspects, the at least one sensor can be an amplitude mode (A-mode) ultrasound sensor, a brightness mode (B-mode) ultrasound sensor, and/or a 3D ultrasound sensor. The at least one target can also have a variety of configurations, and in one embodiment the at least one target can be a light-emitting diode (LED).

In another embodiment, the at least one patch can include at least first and second patches. The first patch can be configured to track movement of a first vertebra, while the second patch can be configured to track movement of a second vertebra in the patient.

In another aspect, a surgical tracking system is provided that includes at least one patch with a patient-facing surface that is configured to be removably attached to a skin surface of a patient. The at least one patch can be configured to track a distance of at least one selectable element within the patient relative to the patch. The system can also include a monitoring system that is configured to track an orientation and a location of the at least one patch relative to the monitoring system.

The system can have a number of variations. For example, the at least one patch can include at least first and second ultrasound sensors. The first ultrasound sensor can be configured to track a first distance between the first ultrasound sensor and a first part of the selectable element, and the second ultrasound sensor can be configured to track a second distance between the second ultrasound sensor and a second part of the selectable element. In another embodiment, the monitoring system can be configured to determine at least one of a total distance from the at least one patch to the selectable element and an axial rotation of the selectable element relative to the at least one patch based on the first distance, the second distance, and the orientation and the location of the at least one patch over time. The monitoring system can also include a navigation camera for viewing the at least one patch.

In some embodiments, the at least one patch can have a plurality of tracking targets on an outward facing surface thereof, and the monitoring system can be configured to track the orientation and the location of the at least one patch relative to the monitoring system based on locations of the plurality of tracking targets over time. The plurality of tracking targets can be light-emitting diodes (LEDs) in some embodiments. In another embodiments, the at least one patch can have at least one ultrasound sensor therein.

In still another aspect, a surgical tracking method is provided that includes applying a first patch to a skin surface of a patient adjacent a spine of the patient. The first patch can have a plurality of ultrasound sensors therein. The method also includes tracking, by the patch, movement over time of the first vertebra relative to the plurality of ultrasound sensors by measuring distances between each of the plurality of ultrasound sensors and the first vertebra. The method also includes tracking, by a monitoring system, an orientation and a location over time of the first patch relative to the monitoring system, and displaying on a surgical display a virtual representation of the orientation and the location of the first vertebra relative to the monitoring system.

The method can include numerous variations. For example, the method can include associating, by the monitoring system, the first patch with a first vertebra in the spine of the patient. It can also include applying at least one additional patch to the skin surface of the patient. The at least one additional patch can have a plurality of ultrasound sensors therein. The method can also include associating, by the monitoring system, the at least one additional patch with at least one additional vertebra in the spine of the patient, and tracking, by the patch, movement over time of the at least one additional vertebra relative to the at least one additional patch by measuring distances between each of the plurality of ultrasound sensors of the at least one additional patch and the at least one additional vertebra. The method can also include tracking, by the monitoring system, an orientation and a location over time of the at least one additional patch relative to the monitoring system. In another embodiment, tracking the orientation and the location of the first patch can include tracking a position over time of a plurality of targets on an outward-facing surface of the first patch using the monitoring system. The method can also include determining an axial rotation and displacement of the first vertebra relative to the first patch over time based on the measurements of distances between each of the plurality of ultrasound sensors and the first vertebra. In another embodiment, the method can include, before applying the first patch, imaging the spine of the patient to identify individual vertebrae. Some embodiments of the method can also include, after applying the first patch, imaging the spine of the patient to identify individual vertebrae and associating, by the monitoring system, the first patch with the first vertebra in the spine of the patient automatically based on the imaging of the spine and placement of one or more fiducials in the first patch that are imposed on the imaging of the spine.

### BRIEF DESCRIPTION OF DRAWINGS

The embodiments described above will be more fully understood from the following detailed description taken in conjunction with the accompanying drawings. The drawings are not intended to be drawn to scale. For purposes of clarity, not every component may be labeled in every drawing. In the drawings:

FIG. 1 is a partial cross-sectional view of one embodiment of a surgical patch placed on a skin surface of a patient over a vertebra;

FIG. 2 is a top down view of an operating room that includes one embodiment of a tracking system with a plurality of patches from FIG. 1 placed on the patient of FIG. 1;

FIG. 3 is a top down partially transparent view of a plurality of patches of FIG. 1 showing sensors therein placed on the patient of FIG. 1;

FIG. 4 is a diagram of one embodiment of a control system architecture;

FIG. 5 is a flow diagram of the tracking system of FIG. 2; and

FIG. 6 is another flow diagram of the tracking system of FIG. 2.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Various exemplary methods, devices, and systems are provided for tracking surgically-relevant elements within a patient, for example tracking one or more vertebra noninvasively during various spinal operations. An orientation of a surgically-relevant element, for example a vertebra, can be tracked within a body of a patient by a patch placed on a skin surface of the patient, while a location of the patch can be tracked within an operating room generally. Because the orientation of the surgically-relevant element to the patch is known and because the location of the patch within the operating room is known (for example, by imaging), these measurements can be combined to allow s determination of the location and orientation of the surgically-relevant element relative to the operating room as a whole. This information can allow surgeons to more accurately operate on the patient because the location and orientation of the element being operated on, such as a vertebra, is known even as movement of the surgically-relevant element occurs during the operation. When using various surgical instruments, such as robotic surgical systems, the location and orientation of the surgically-relevant element relative to the operating room as a whole can be used to more accurately maneuver the surgical instruments, such as those attached to and/or operated by robotic surgical systems.

An exemplary non-invasive tracking or reference mechanism can be in the form of at least one patch having a patient-facing surface configured to be removably attached to an outer skin surface of a patient, and having an outward-facing surface. The patch can be configured to track a distance between the patch and at least one selectable element, such as a vertebra, within a patient. The patch can also be configured to be tracked by an external monitoring system that tracks its orientation and location relative to the monitoring system. As such, the patch can include at least one sensor therein that is configured to measure a relative distance between the sensor and the selectable element within the patient, as well as at least one target that is positioned on the patch and that is configured to be tracked by the monitoring system. The mechanism thus allows for correlation of a selectable element with respect to the patch, and the patch itself to be tracked within an operating space. This allows each selectable element to be individually tracked in a 3D coordinate system and optionally visualized in real time. When one or more vertebra are being tracked, the tracking and optional visualization thus reflects the actual anatomical situation of the spine. The mechanism also allows for compensation of any skin motion of a patient while still providing real-time tracking of multiple vertebral bodies.

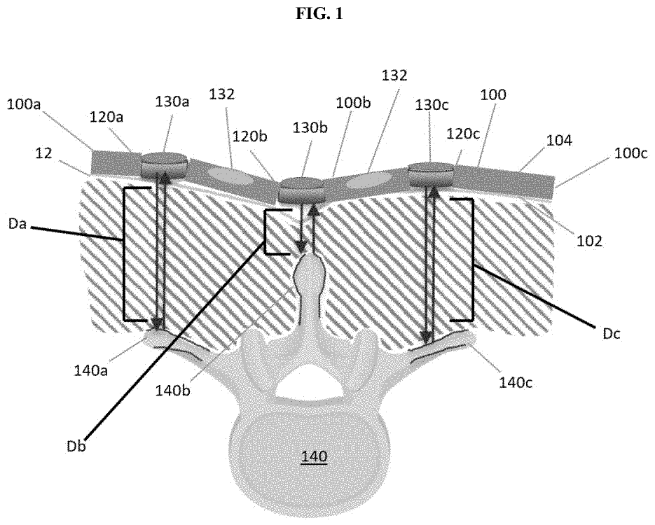

FIG. 1 illustrates one embodiment of a patch 100 that is configured to be used in determining a relative distance to a selectable element 140, such as a vertebra, and that is configured to be tracked by an external monitoring system, therefore acting as a reference device. The patch 100 is configured to be associated with the same selectable element 140 throughout an operation. Thus, the patch 100 is configured to allow determination of a pose or orientation and location of the selectable element 140 within the patient with respect to an external reference point. As shown, the patch 100 has a patient-facing surface 102 that is configured to be removably attached to a skin surface 12 of a patient 10, an outward-facing surface 104 that is configured to face away from the patient 10, and at least one sensor 120a, 120b, 120c contained therein or on the patient-facing surface 102 thereof. The illustrated patch 100 has three sensors, however the patch can include any number of sensors.

As discussed below, the patch 100 in FIGS. 1-3 is made of a flexible material. However, the patch 100 can be flexible or rigid and can be made from a variety of different materials known in the art, such as medical grade polymers, various stretchable materials, elastomers, silicone, etc. The patch 100 can also be attached to the skin of the patient through a variety of known means, such as through a removable adhesive. The patch 100 can also have a variety of different predetermined or known sizes and shapes depending on the desired use, such as rectangular, square, circular, an anatomical shape, to better track a particular selectable element within a patient. For example, the patch 100 can be sized and shaped for use on either the lumbar or cervical spine, such as being shaped to correspond to one or more lumbar or cervical vertebrae. The patch 100 can also be part of a robotic surgical system or a computer-assisted surgical system.

The at least one sensor 120a, 120b, 120c can be configured to sense and/or measure a relative distance between the at least one sensor and at least part of the selectable element 140, such as a vertebra as illustrated in FIGS. 1-3. Each sensor 120a, 120b, 120c can be configured to take multiple measurements over time such that a movement of the selectable element 140 over time can be measured based on relative changes in distance measurements by the sensor, discussed in detail below. While the sensors 120a, 120b, 120c are discussed herein as measuring a distance, sensors may either measure a distance directly or provide various readings, data, information, etc., to a secondary element, such as a monitoring system 200 and/or a control system 300 discussed below.

The at least one sensor 120a, 120b, 120c can take a variety of forms, however the illustrated sensors are ultrasound sensors. Each sensor 120a, 120b, 120c is configured to broadcast a signal into the patient and receive an echo of its original signal from the selectable element 140 to determine distance based on a time between sent and received signals, as illustrated in FIG. 1 by arrows showing the broadcast and return signals. However, the sensor is not limited to such a distance-measuring mechanism. Each illustrated sensor 120a, 120b, 120c can be a type of ultrasound sensor or ultrasound transducer that is a transceiver, being able to both transmit and receive ultrasound. However, the patch 100 can include various combinations of transmitter, receiver, and transceiver ultrasound sensors in other embodiments. Furthermore, the sensor 120a, 120b, 120c can be a variety of different types of ultrasound sensors, such as an amplitude mode (A-mode) ultrasound sensor, a brightness mode (B-mode) ultrasound sensor, a 3D ultrasound sensor, or various other piezoelectronic ultrasonic transducers. Each sensor 120a, 120b, 120c can have an individual power source, can share a collective power source, or can be a passive sensor. The sensors can also be wired or can connect wirelessly to the patch 100 and/or a secondary element, such as a monitoring system 200 and/or a control system 300 discussed below, and they can provide a variety of information and/or data thereto, such as sensor readings. The sensors can also take measurements at selectable times, at periodic times, or in real time. Additionally, sensors or transducers in some embodiments can be used to construct virtual 3D volume models of any sensed internal elements of the patient 10, for example by creating one or more volumetric image datasets of a relevant surgical site in the patient 10. While patches and sensors are discussed herein that are removably attached to the skin surface of the patient, non-contact sensing can also be used in some embodiments, in which either one or more sensors or the entire patch can perform sensing of one or more selectable elements without requiring the patch and/or the one or more sensors to directly contact the skin surface and/or the patient entirely. As a non-limiting example, one or more ultrasound sensors can be separated from the patient's skin surface by air, and the sensor(s) can focus ultrasound beams at the skin surface that can transmit through the air and pass across the air-skin surface boundary to provide sensing within the patient.

The patch 100 illustrated in FIGS. 1-3 includes three sensors 120a, 120b, 120c that are configured to individually measure a relative distance between each of the plurality of sensors 120a, 120b, 120c and a corresponding individual part of the selectable element 140. Each sensor 120a, 120b, 120c can be fixed in the patch 100 such that the sensor's position relative to the patch 100 and relative to the skin surface 12 of the patient do not change over time, providing a consistent reference point at the patch 100. In such an example, the patch 100 can be a flexible material that is stretched and adhesively fixed along the outer skin surface 12 of the patient 10, and the sensors 120a, 120b, 120c can be embedded therein such that, as the patient 10 is moved during surgery, the patch 100 remains stretched and fixed to the same location on the patient's skin 12, and the sensors 120a, 120b, 120c remain secured to the same location within the patch 100. By comparing distances measured by each of the plurality of sensors 120, an orientation and an overall distance of the selectable element 140 within the patient relative to the patch 100 can be determined. For example, each of the plurality of sensors 120a, 120b, 120c is configured to measure a relative distance Da, Db, Dc to a corresponding point 140a, 140b, 140c on the selectable element 140, as illustrated in FIG. 1. The plurality of sensors 120a, 120b, 120c are configured to take multiple measurements over time such that rotation and movement of the selectable element 140 relative to the patch 100 can be measured over time. Measurements can be used to calculate a variety of orientations and movements, for example an axial orientation about an axis extending along the patient's spine and through the selectable element 140, a relative axial rotation of the axial orientation over time, an overall distance between the patch 100 and the selectable element 140 based on the plurality of distance measurements from the plurality of sensors 120a, 120b, 120c, and a relative movement of the element 140 by comparing the overall distance over time.

For example, the measured distances Da, Dc each represent distances from the sensors 120a, 120c to the points 140a, 140c of lateral elements on the selectable element 140, such as lateral masses of the illustrated vertebra, and the measured distance Db represents the distance from the sensor 120b to a point 140b on a center of the selectable element 140, such as the spinous process of the illustrated vertebra. Using these distances, the axial orientation of the element 140 at the time of measurement can be determined by comparing the measured distances Da, Db, Dc to each other. For instance, if the distance Da is greater than the distance Dc, then the element 140 is oriented with the point 140a of the corresponding lateral mass rotated away from the patch 100 and the point 140c of the corresponding lateral mass rotated toward the patch 100. If the distance Da is equal to the distance Dc, then the element 140 is oriented approximately parallel with the patch 100 (as illustrated in FIG. 1). When a plurality of measurements are taken over time, the relative axial rotation of the element 140 over time can be determined by comparing changes in the measured distances Da, Db, Dc to each other over time. If the distance Da is initially greater than the distance Dc, then equal to the distance Dc, and finally less than the distance Dc, then the element 140 will have rotated over time with the point 140a of the corresponding lateral mass rotating toward the patch 100 and the point 140c of the corresponding lateral mass rotating away from the patch 100. The overall distance between the patch 100 and the element 140 at the time of measurement can be determined by measuring the distances Da, Db, Dc and knowing the fixed location of the sensors 120a, 120b, 120c in the patch 100 and the size and shape of the patch 100. Movement of the element 140 relative to the overall patch 100 over time can be determined by comparing changes in the distances Da, Db, Dc to themselves over time. If each of the measured distances Da, Db, Dc at a first time is greater than the measured distances Da, Db, Dc at a second time, the element 140 as a whole will have moved toward the patch 100. Rotation and movement of the element 140 can thus be tracked over time to provide an accurate, 3-dimensional understanding of the location of the element 140 relative to the patch 100 at any given time.

As noted above, the patch 100 illustrated in FIGS. 1-3 contains three sensors 120a, 120b, 120c, however any number of sensors can be included, such as between 1 sensor and 1000 sensors and more preferably between 2-10, depending on the type of sensor used and a desired level of detail in measurements. For example, in various embodiments, 2 to 5 3D ultrasound sensors can be used, 3 to 10 A-mode ultrasound sensor can be used, 3 to 5 B-mode ultrasound sensors can be used, etc. Including more sensors can produce additional measurements of distances over time and thus provide a clearer understanding of the orientation and position of the selectable element. Additionally, while the illustrated sensors 120a, 120b, 120c are arranged linearly, a variety of spatial arrangements can be used depending on desired measurements. Various flat, 2-dimensional spatial arrangements, such as an array or matrix of sensors, can be used to provide additional measurements and thus a clearer understanding of orientation and position. For example, if an array or matrix of sensors is used, orientations across other axes and relative rotations can also be determined, such as along an axis perpendicular to the axis extending along the patient's spine. Thus, the plurality of sensors 120a, 120b, 120c can have a variety of different arrangements on the patch as determined by the desired use, such as rectangles, squares, circles, anatomical shapes to better track a particular selectable element within a patient, etc. While the arrangement of the plurality of sensors 120a, 120b, 120c with respect to each other and the patch 100 can vary in different embodiments, the plurality of sensors 120a, 120b, 120c can be fixed within the patch 100 such that the arrangement is known before use and does not change during use. The sensors 120a, 120b, 120c can be made from a variety of different materials known in the art, such as medical grade metals, polymers, various stretchable materials, elastics, etc.

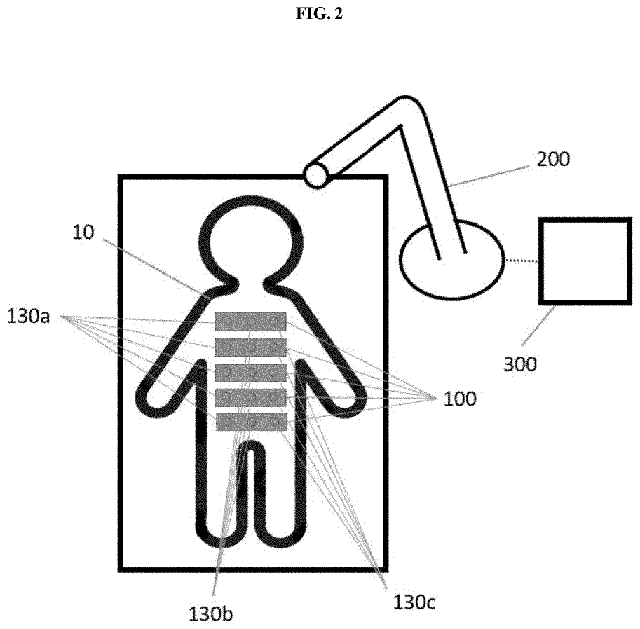

As previously mentioned, the patch 100 is also configured to be tracked by the external monitoring system 200 to provide an orientation and a location of the patch 100 with respect to the external monitoring system 200. For example, FIGS. 1 and 2 illustrate three targets 130a, 130b, 130c on the outward-facing surface 104 of the patch 100 that is configured to be tracked by the external monitoring system 200. The illustrated targets 130a, 130b, 130c are placed on top of the corresponding sensors 120a, 120b, 120c so that, as the location and movement over time of each target 130a, 130b, 130c is tracked, the location and movement of the corresponding sensor 120a, 120b, 120c can be determined over time because it is coupled to the target 130a, 130b, 130c. However, each target 130a, 130b, 130c can be fixed at various locations on the outward-facing surface 104 of the patch 100, and each target 130a, 130b, 130c can be configured to be tracked by the monitoring system 200 through a variety of different ways, as discussed in more detail below.

While the targets 130a, 130b, 130c can be a light-emitting diodes (LED), each target can take a variety of forms, such as printed and/or reflective symbols or geometric shapes, fluorescent or ultraviolet lights, sensors, various radio-frequency identification (RFID) tags or various transmitting tags, printed gridlines or repeating patterns, gyroscopes, accelerometers, etc. Additionally, in other embodiments, each target can be placed within the patch or on various other surfaces of the patch, etc. The patch 100 can also include various combinations of different types of targets. Each target can have an individual power source, can share a collective power source, or can be a passive target. Each target can optionally be wired or can connect wirelessly to the patch 100 and/or an external monitoring system depending on the target used, and each target can also broadcast its location at selectable points, at periodic points, or in real time depending on the type of target used.

The patch 100 illustrated in FIGS. 1-3 includes three targets 130a, 130b, 130c that are configured to be individually tracked by the external monitoring system 200, and by comparing a location of each target 130a, 130b, 130c relative to the monitoring system 200 and relative to each other target 130a, 130b, 130c (as tracked by the monitoring system 200), an orientation and an overall location of the patch 100 relative to the external monitoring system 200 can be determined, as illustrated in FIG. 2. The targets 130a, 130b, 130c can be tracked by the monitoring system 200 in a variety of ways. For example, in one embodiment, each target 130a, 130b, 130c can be fixed in the patch 100 such that the target's position relative to the patch 100 and relative to the skin surface 12 of the patient do not change over time. In such an example, the patch 100 can be a flexible material that is stretched and adhesively fixed along the outer skin surface 12 of the patient 10 as noted above, and the targets 130a, 130b, 130c can be fixed on the outwardly-facing surface 104 such that, as the patient 10 is moved during surgery, the patch 100 remains stretched and fixed to the same location on the patient's skin 12 and the targets 130a, 130b, 130c remain fixed on the same location on the outward-facing surface 104 of the patch 100. For example, measurements can be used to calculate an axial orientation about an axis extending parallel to the patient's spine and along the patient's skin, a relative axial rotation of the axial orientation over time, an overall location of the patch 100 relative to the external monitoring system 200 based on the plurality of tracked locations from the plurality of targets 130a, 130b, 130c, and a relative movement of the patch 100 by comparing the overall location over time.

While the targets 130a, 130b, 130c can be configured to be tracked in a variety of means, in one embodiment, the targets 130a, 130b, 130c are configured to have their images taken repeatedly by the monitoring system 200. The targets 130a, 130b, 130c can be configured to have an initial image taken by the monitoring system 200 that can then be used as a starting reference. The initial orientation and location of the patch 100 can be determined by analyzing the initial image because the targets 130a, 130b, 130c are placed on the patch 100 in fixed, known locations (providing points of reference on the patch 100 itself) and because the size and shape of the patch 100 is known (allowing determination of orientation). The targets 130a, 130b, 130c can be configured to be imaged on an ongoing basis by the monitoring system 200 such that, each time a new image of the targets 130a, 130b, 130c is acquired, a new location of the targets 130a, 130b, 130c can be determined relative to each of their previous locations and relative to each other. In the illustrated example, targets 130a, 130c are each placed on lateral sides 100a, 100c of the patch 100 above lateral elements of the selectable element 140, such as lateral masses of the illustrated vertebra, and target 130b is placed on a center 100b of the patch 100 above a center of the selectable element 140, such as the spinous process of the vertebra, as illustrated in FIG. 1. Using these targets, the axial orientation of the patch 100 at the time of each image can be determined by, for example, comparing the sizes and/or orientations of the targets 130a, 130b, 130c in the image. For instance, if target 130a is smaller than target 130c in the image, then the patch 100 is oriented with the lateral side 100a of the patch 100 rotated away from the monitoring system 200 and the lateral side 100c rotated toward the monitoring system 200. If the targets 130a, 130b, 130c are approximately equal in the image, then the patch 100 is approximately oriented to directly face the monitoring system 200. When a plurality of images are taken over time, the relative axial rotation of the patch 100 over time can be determined by comparing changes in positions and sizes of the targets 130a, 130b, 130c in the images to each other over time. If target 130a is initially smaller than target 130c, then equal to target 130c, and finally larger than target 130c in successive images, then the patch 100 will have rotated over time with the side 100a rotating toward the monitoring system 200 and the side 100c rotating away from the monitoring system 200. The overall distance between the patch 100 and the imaging system 200 at the time of imaging can be determined by imaging the targets 130a, 130b, 130c and knowing the fixed location of the targets 130a, 130b, 130c in the patch 100 (providing a real-world reference measurement) and the size and shape of the patch 100. For example, if a distance between each target 130a, 130b, 130c is a known value, a scale can be determined for any image taken in which the targets 130a, 130b, 130c are visible by assuming that the distance between each target 130a, 130b, 130c in the image is the known value and applying that same scale to the rest of the image. Movement of the patch 100 relative to the monitoring system 200 over time can be determined by comparing changes in locations of the targets 130a, 130b, 130c in successive images over time. For example, if all of the targets 130a, 130b, 130c in a second image have shifted to the right when compared to the targets 130a, 130b, 130c from a first image, then the patch 100 as a whole will have moved right relative to the monitoring system 200. Rotation and movement of the patch 100 can thus be tracked over time to provide an accurate, 3-dimensional understanding of the location of the patch 100 relative to the monitoring system 200 at any given time.

While specific tracking mechanisms were discussed for targets 130a, 130b, 130c, tracking mechanisms of the monitoring system 200 are not limited to these approaches. For example, in other embodiments, the targets can represent known geometric patterns, 3-dimensional shapes, orientations, patterns of light, etc. placed on the patch at known locations, and the monitoring system can track what portions of the targets are visible to the monitoring system in each image through successive images taken over time. As the visible portions of the targets change, shift, etc. over time, the monitoring system can determine an orientation and/or location of each target and consequently an orientation and/or location of the patch as a whole because the targets are placed on fixed locations of the patch 100. In still other embodiments, the targets can represent various elements that broadcast or transmit their locations, such as through various radio frequencies, to the monitoring system, which tracks their distance and direction away from the monitoring system. By comparing these received signals relative to each other over time, the monitoring system can thus track movement and approximate orientation of the targets and consequently the patch.

While the patch 100 illustrated in FIGS. 1-3 contains three targets 130a, 130b, 130c, any number of targets can be included, such as between 1 target and 100 targets and more preferably from 3 to 10, depending on the type of target used and a desired level of detail in measurements. For example, including more targets can produce additional location measurements over time and thus provide a clearer understanding of the orientation and location of the patch 100 relative to the external monitoring system 200. Additionally, while the illustrated targets 130a, 130b, 130c are arranged linearly, a variety of spatial arrangements can be used depending on desired measurements. Various flat, 2-dimensional spatial arrangements, such as an array or matrix of targets, can be used to provide additional measurements and thus a clearer understanding of orientation and location, as discussed above. If an array or matrix of targets is used, orientations across other axes and relative rotations can also be determined, such as along an axis perpendicular to the axis extending along the patient's skin. The targets 130a, 130b, 130c can have a variety of different arrangements as determined by the desired use, such as rectangles, squares, circles, anatomical shapes to better track a particular selectable element within a patient, etc. While the targets 130a, 130b, 130c are disposed on an outward-facing surface, targets can be disposed within the patch 100 or on another surface depending on the type of target used, such as RFID tags or various transmitting tags. The targets 130a, 130b, 130c can be made from a variety of different materials known in the art, such as medical grade metals, polymers, various stretchable materials, elastics, etc.

Furthermore, the patch 100 illustrated in FIGS. 1-3 includes one or more fiducials 132 for intraoperative registration, as discussed below. The one or more fiducials 132 are objects viewable or detectable by the monitoring system 200 that can subsequently be shown in images, displays, etc. produced by the monitoring system 200 for use as one or more points of reference or measurement. The one or more fiducials 132 can either be placed into or on an external surface of the patch 100. While fiducials 132 are provided on the patch 100, patch(es) in other embodiments can have no fiducials.

As noted, the patch 100 can be tracked by a monitoring system, such as the monitoring system 200 illustrated in FIG. 2. Specifically, the monitoring system 200 is configured to track an orientation and a location of the at least one patch 100 relative to the monitoring system 200. It is configured to track the one or more targets 130a, 130b, 130c on the outward-facing surface 104 of the patch 100 to determine the relative location of each target 130a, 130b, 130c to the monitoring system 200 and the orientation of the patch 100 with respect to the monitoring system 200 because each target's position relative to the patch 100 does not change over time. The monitoring system 200 can be configured to track the patch 100 in the form of a plurality of data points representing a defined shape of the patch 100 and can be configured to update the plurality of data points during tracking based on changes to the orientation and/or location. The monitoring system 200 can have a variety of configurations and can include various components, such as a navigation camera used for surgery. Depending on the type of target used, the monitoring system 200 can be configured to directly visualize the patch through one or more cameras and/or other optical navigation approaches, such as when LED targets are being used. However, the monitoring system 200 can use active tracking, passive tracking, or some combination, and it can use magnetic tracking of patches in some embodiments, such as using electromagnetic navigation. The monitoring system 200 can be part of a robotic surgical system, part of a computer-assisted surgical system, or a stand-alone device.

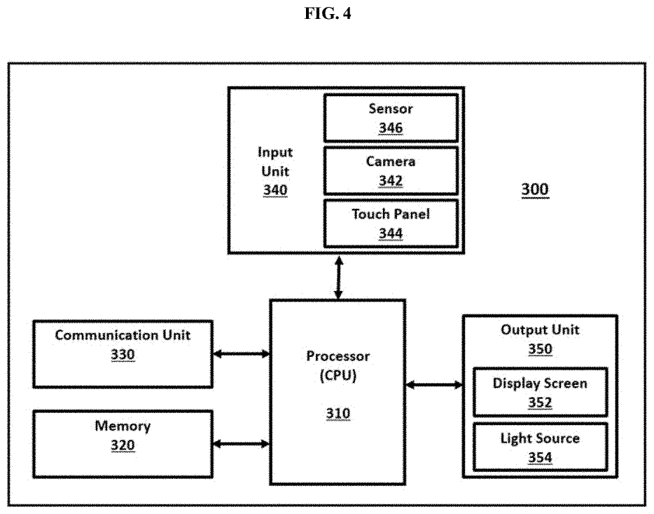

The control system or processor 300 is configured to assist in calculating the orientation and location of the at least one patch 100 relative to the monitoring system 200 based on data gathered by the monitoring system 200. It is also configured to assist in calculating an orientation and distance(s) of the at least one selectable element 140 within the patient relative to the patch 100 and/or one or more sensors 120a, 120b, 120c. In both situations, the control system 300 can be configured to calculate results both at a single point in time, periodically, or continuously over a period of time. The control system 300 can either be part of the monitoring system 200, can be incorporated in one or more patches 100 and/or sensors 120a, 120b, 120c, can be part of a robotic surgical system, can be a separate component, or some combination of the preceding. In some embodiments, it can also communicate with at least one of the monitoring system 200, the patch 100, and/or one or more of the sensors 120a, 120b, 120c, either directly or indirectly and either wirelessly or through wired connections. FIG. 4 illustrates a diagrammatic view of an exemplary device architecture of the control system 300.

As shown in FIG. 4, the control system 300 may contain multiple components, including, but not limited to, an internal processor (e.g., central processing unit (CPU) 310, a memory 320, a wired or wireless communication unit 330, one or more input units 340, and one or more output units 350. It should be noted that the architecture depicted in FIG. 4 is simplified and provided merely for demonstration purposes. The architecture of the control system 300 can be modified in any suitable manner as would be understood by a person having ordinary skill in the art, in accordance with the present claims. Moreover, the components of the control system 300 themselves may be modified in any suitable manner as would be understood by a person having ordinary skill in the art, in accordance with the present claims. Therefore, the device architecture depicted in FIG. 4 should be treated as exemplary only and should not be treated as limiting the scope of the present disclosure.

The internal processor 310 is capable of controlling operation of the control system 300 and/or the monitoring system 200 depending on whether the control system 300 and the monitor system 200 are combined or separate. More specifically, the processor 310 may be operable to control and interact with multiple components associated with the control system 300, as shown in FIG. 4. For instance, the memory 320 can store program instructions that are executable by the internal processor 310 and data. The process described herein may be stored in the form of program instructions in the memory 320 for execution by the internal processor 310. The communication unit 330 can allow the control system 300 to transmit data to and receive data from one or more external devices via a communication network. The input unit 340 can enable the control system 300 to receive input of various types, such as audio/visual input, user input, data input, and the like. To this end, the input unit 340 may be composed of multiple input devices for accepting input of various types, including, for instance, one or more cameras 342 (i.e., an “image acquisition unit”), touch panel(s) 344, microphone(s) (not shown), sensors 346, one or more buttons or switches (not shown), and so forth. The input devices included in the input 340 may be manipulated by a user. Notably, the term “image acquisition unit,” as used herein, may refer to the camera 342, but is not limited thereto. For example, the image acquisition unit can be the monitoring system 200 or a part thereof. The output unit 350 can display information on the display screen 352 for a user to view. The display screen 352 can also be configured to accept one or more inputs, such as a user tapping or pressing the screen 352, through a variety of mechanisms known in the art, and the output unit 350 may further include a light source 354. In some embodiments, the output unit 350 can be configured to send any processing results to various systems, such as a robotic surgical system or a computer-assisted surgical system. The control system 300 and/or the monitoring system 200 can also be configured to calculate distance measurements based on sensor readings from the one or more sensors 120a, 120b, 120c. The control system 300 and/or the monitoring system 200 can thus be configured to track and model the at least one selectable element 140 and/or the patch 100 as pluralities of data points representing a defined for the selectable element 140 and/or the patch 100 and can be configured to update each of the pluralities of data points during tracking based on changes to the orientation(s) and/or location(s) as a result of the various calculations discussed herein. These pluralities of data points can be stored and updated on various different types of memory, such as memory 320.

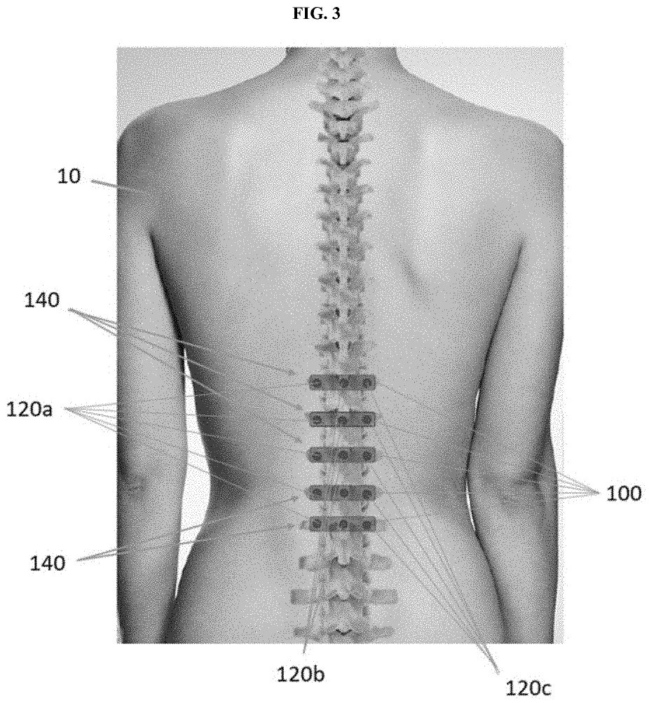

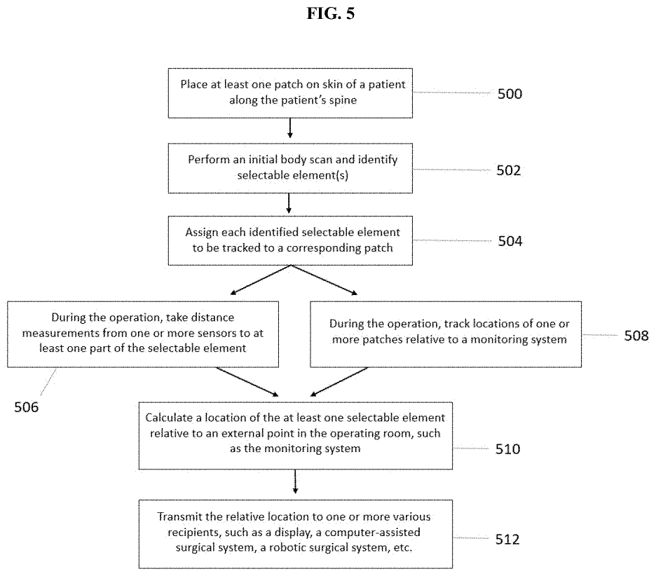

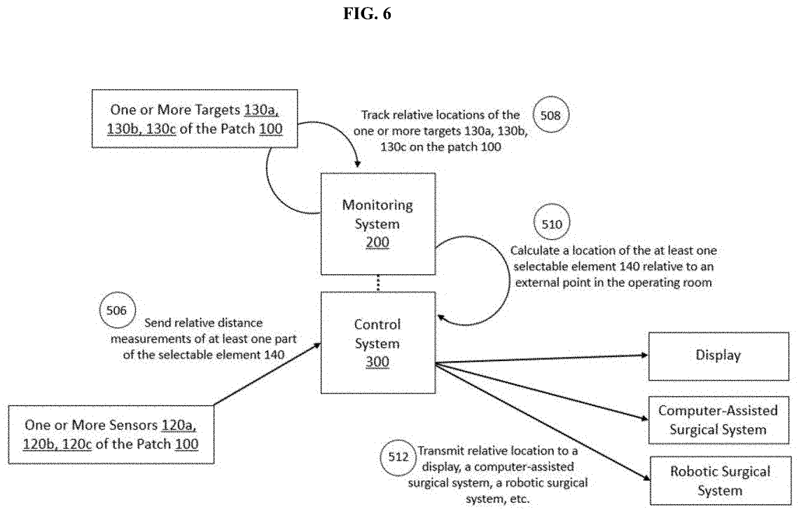

FIGS. 5 and 6 illustrate simplified flow diagrams of the tracking mechanism discussed above in use. During an operation, one or more patches 100 are attached to the patient 10 at step 500. The patient can be positioned on their stomach or side, and the patient-facing surface 102 of each patch 100 is removably attached to the skin 12 of the patient along the patient's spine so that the outward-facing surface 104 faces away from the patient 10. Initial placement and/or positioning of the patch(es) 100 on the skin surface 12 of the patient 10 can be performed in a variety of ways. For example, each patch 100 can be placed over a vertebral element of interest, as illustrated in FIG. 3. In such an example, alignment of each patch 100 can be arranged so that the sensors 120a, 120c are generally aligned with the points 140a, 140c of the lateral elements (lateral masses) on the selectable element 140, and the sensor 120b is generally aligned with the point 140b on the center (spinous process) of the selectable element 140. Initial alignment can be guided by various probing, preoperative imaging (discussed below), surgical experience, etc.

To determine initial locations of one or more selectable elements 140, such as individual vertebra, an initial body scan can be performed on the patient 10 at step 502, such as an intraoperative 3D scan like an 0-arm scan, an AIRO scan, a 3D fluoroscopy scan, etc. While the one or more patches 100 can be attached to the patient 10 before the initial body scan is performed, in some embodiments, preoperative imaging can first be performed, the one or more patches 100 can then be attached based on guidance from the preoperative imaging, and any additional body scanning can subsequently be performed to determine initial locations of selectable elements 140 to increase the accuracy of initial patch placement. Preoperative imaging can be performed using known methods, such as by using an intraoperative 3D scan discussed above, or can be performed by the one or more patches 100 directly.

Once the initial body scan is performed, the results can be segmented to allow identification and separation of individual selectable elements 140, such as individual vertebra. As such, each identified selectable element 140 can be uniquely assigned to an individual corresponding patch 100 at step 504, which allows each selectable element 140 to be independently tracked separate from any other selectable element 140. These unique assignments can be provided to the monitoring system 200 and/or the control system 300 so that, during use, the system(s) can monitor movement of each unique selectable element 140 and distinguish signals and/or information for each unique patch 100 from any other patches 100 used. In some embodiments, the one or more patches 100 can be removed and realigned as needed to ensure that each corresponding sensor 120a, 120b, 120c is generally aligned with each corresponding point 140a, 140b, 140c of the corresponding selectable element 140.

In some embodiments, the one or more fiducials 132 of the one or more patches 100 can be used for intraoperative registration of one or more selectable elements 140. In such embodiments, patch(es) 100 can be attached to the skin 12 of the patient 10 that contain one or more fiducials 132 therein, and the initial body scan can be performed and provided to the monitoring system 200. The monitoring system 200 can detect the fiducials 132 in the patches 100, and the initial body scan can provide images to the monitoring system 200 that can subsequently have relative positions of the fiducials 132 in relation to the newly imaged selectable elements 140 provided thereon. As such, the monitoring system 200 can identify the selectable elements 140 and the patches 100, at least based in part on the fiducials 132. The monitoring system 200 can then correlate each patch 100 with the corresponding selectable element 140. The correlation process can either be automatically performed by the monitoring system 200 or manually performed by a surgeon.

During the operation, one or more sensors 120a, 120b, 120c within each patch 100 measures a relative distance Da, Db, Dc between each sensor 120a, 120b, 120c and each point 140a, 140b, 140c of the selectable element 140 at step 506, either once, periodically, or continuously. Based on comparing the distances Da, Db, Dc to each other and to themselves over time, orientation and/or movement of the selectable element 140 is tracked over time relative to the patch 100, as discussed in detail above. The calculations and analysis can be performed by the monitoring system 200 and/or the control system 300.

While the sensors 120a, 120b, 120c track the selectable element 140 within the body relative to the patch 100, the monitoring system 200 tracks the location of the patch 100 at step 508, for example by tracking the locations of the one or more targets 130a, 130b, 130c on the outward-facing surface 104 of each patch 100 by taking a series of images of the targets 130a, 130b, 130c. Based on changes in the target locations in the series of images, the orientation and/or location of each target 130a, 130b, 130c and consequently each patch 100 relative to the monitoring system 200 is tracked over time, as discussed above in detail. The calculations and analysis can also be performed by the monitoring system 200 and/or the control system 300.

Because each patch 100 measures an orientation and/or movement of a selectable element 140 relative to the patch 100, and because the monitoring system 200 measures the orientation and/or location of each patch 100 relative to the monitoring system 200, the monitoring system 200 and/or the control system 300 can thus calculate an accurate location and orientation of each selectable element 140 within the patient 10 over time relative to a reference point outside of the patient in the operating room at step 510, such as the monitoring system 200. As such, accurate navigation is possible during the operation because an accurate understanding of the location and orientation of the selectable element 140 within the patient 10 is possible non-invasively and in real time. This navigation is thus possible even when intervertebral relationships change due to various common factors, such as force being applied, disc removal, patient or instrument movement, etc., rather than being limited to estimating locations of each selectable element 140 based on inaccurate data.

This orientation and location information can be provided to the surgeon and/or a surgical system through a variety of means at step 512, for example by being displayed on various displays for the surgeon, by being modeled as a virtual 3-dimensional image in real time or periodically, by being provided to a computer-assisted surgical system, by being provided to a robotic surgical system, etc. As noted above, however, the monitoring system 200 and/or the control system 300 can be directly incorporated into various computer-assisted surgical systems and/or robotic surgical systems in other embodiments. In some embodiments, the overall tracking system disclosed herein can be used to perform preoperative imaging and intraoperative registration of instruments and apparatuses directly.

All of the devices disclosed herein, such as the various patches, sensors, targets, instruments, tools, etc., can be designed to be disposed of after a single use, or they can be designed to be used multiple times. In either case, however, the devices can be reconditioned for reuse after at least one use. Reconditioning can include any combination of the steps of disassembly of the devices, followed by cleaning or replacement of particular pieces, and subsequent reassembly. In particular, the devices can be disassembled, and any number of the particular pieces or parts of the device can be selectively replaced or removed in any combination. Upon cleaning and/or replacement of particular parts, the devices can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

It is preferred that devices disclosed herein be sterilized before use. This can be done by any number of ways known to those skilled in the art including beta or gamma radiation, ethylene oxide, steam, and a liquid bath (e.g., cold soak). An exemplary embodiment of sterilizing a device including internal circuitry is described in more detail in U.S. Pat. Pub. No. 2009/0202387 filed Feb. 8, 2008 and entitled “System And Method Of Sterilizing An Implantable Medical Device.” It is preferred that device, if implanted, is hermetically sealed. This can be done by any number of ways known to those skilled in the art.

Further, in the present disclosure, like-named components of the embodiments generally have similar features, and thus within a particular embodiment each feature of each like-named component is not necessarily fully elaborated upon. Additionally, to the extent that linear or circular dimensions are used in the description of the disclosed systems, devices, and methods, such dimensions are not intended to limit the types of shapes that can be used in conjunction with such systems, devices, and methods. A person skilled in the art will recognize that an equivalent to such linear and circular dimensions can easily be determined for any geometric shape. Sizes and shapes of the systems and devices, and the components thereof, can depend at least on the anatomy of the subject in which the systems and devices will be used, the size and shape of components with which the systems and devices will be used, and the methods and procedures in which the systems and devices will be used.

Additionally, it is understood that one or more of the systems and methods herein, or aspects thereof, may be executed by at least one processor. The processor may be implemented in various devices, as described herein. A memory configured to store program instructions may also be implemented in the device(s), in which case the processor can be specifically programmed to execute the stored program instructions to perform one or more processes, which are described further herein. Moreover, it is understood that the methods may be executed by a specially designed device, a mobile device, a computing device, etc., comprising the processor, in conjunction with one or more additional components, as described in detail herein.

Furthermore, the systems and methods, or aspects thereof, of the present disclosure may be embodied as non-transitory computer readable media on a computer readable medium containing executable program instructions executed by the processor. Examples of the computer readable mediums include, but are not limited to, ROM, RAM, compact disc (CD)-ROMs, magnetic tapes, floppy disks, flash drives, smart cards, and optical data storage devices. The computer readable recording medium can also be distributed in network-coupled computer systems so that the computer readable media is stored and executed in a distributed fashion, for example by a cloud-based system, a telematics server, a Controller Area Network (CAN), etc. One skilled in the art will appreciate further features and advantages of the described devices and methods based on the above-described embodiments. Accordingly, the present disclosure is not to be limited by what has been particularly shown and described, except as indicated by the appended claims. All publications and references cited herein are expressly incorporated herein by reference in their entirety.

What is claimed is:

1. A surgical tracking device, comprising:
at least one patch having a patient-facing surface configured to be removably attached to skin of a patient, an outward-facing surface, and at least one sensor therein configured to sense a distance between the at least one sensor and at least one selectable vertebra within the patient; and
at least one target on the patch, a relative orientation and location of the at least one target being configured to be tracked by an external monitoring system.

2. The surgical tracking device of claim 1, wherein the at least one sensor comprises at least one ultrasound sensor.

3. The surgical tracking device of claim 1, wherein the at least one patch comprises at least first and second patches, the first patch being configured to track movement of a first vertebra, and the second patch being configured to track movement of a second vertebra in the patient.

4. The surgical tracking device of claim 1, wherein the at least one sensor of the at least one patch is configured to track at least one of a total distance from the at least one patch to the at least one vertebra and an axial rotation of the at least one vertebra relative to the at least one patch over time.

5. The surgical tracking device of claim 1, wherein the at least one target comprises a light-emitting diode (LED).

6. The surgical tracking device of claim 1, wherein the at least one patch comprises a stretchable ultrasound patch and the at least one sensor comprises an array of piezoelectronic ultrasonic transducers.

7. The surgical tracking device of claim 1, wherein the at least one sensor is selected from the group consisting of an amplitude mode (A-mode) ultrasound sensor, a brightness mode (B-mode) ultrasound sensor, and a 3D ultrasound sensor.

8. A surgical tracking system, comprising:
at least one patch having a patient-facing surface configured to be removably attached to a skin surface of a patient, the at least one patch being configured to track a distance of at least one selectable element within the patient relative to the patch; and
a monitoring system configured to track an orientation and a location of the at least one patch relative to the monitoring system.

9. The surgical tracking system of claim 8, wherein the at least one patch includes at least first and second ultrasound sensors, the first ultrasound sensor being configured to track a first distance between the first ultrasound sensor and a first part of the selectable element, and the second ultrasound sensor being configured to track a second distance between the second ultrasound sensor and a second part of the selectable element.

10. The surgical tracking system of claim 9, wherein the monitoring system is configured to determine at least one of a total distance from the at least one patch to the selectable element and an axial rotation of the selectable element relative to the at least one patch based on the first distance, the second distance, and the orientation and the location of the at least one patch over time.

11. The surgical tracking system of claim 8, wherein the monitoring system comprises a navigation camera for viewing the at least one patch.

12. The surgical tracking system of claim 8, wherein the at least one patch has a plurality of tracking targets thereon, and the monitoring system is configured to track the orientation and the location of the at least one patch relative to the monitoring system based on locations of the plurality of tracking targets over time.

13. The surgical tracking system of claim 12, wherein the plurality of tracking targets are light-emitting diodes (LEDs).

14. The surgical tracking system of claim 8, wherein the at least one patch has at least one ultrasound sensor therein.

15. A surgical tracking method, comprising:
applying a first patch to a skin surface of a patient adjacent a first vertebra of a spine of the patient, the first patch having a plurality of ultrasound sensors therein;
tracking, by the patch, movement over time of the first vertebra relative to the plurality of ultrasound sensors by measuring distances between each of the plurality of ultrasound sensors and the first vertebra;
tracking, by a monitoring system, an orientation and a location over time of the first patch relative to the monitoring system; and
displaying on a surgical display a virtual representation of the orientation and the location of the first vertebra relative to the monitoring system.

16. The surgical tracking method of claim 15, further comprising:
associating, by the monitoring system, the first patch with the first vertebra in the spine of the patient;
applying at least one additional patch to the skin surface of the patient, the at least one additional patch having a plurality of ultrasound sensors therein;
associating, by the monitoring system, the at least one additional patch with at least one additional vertebra in the spine of the patient;
tracking, by the patch, movement over time of the at least one additional vertebra relative to the at least one additional patch separate from the first patch and the first vertebra by measuring distances between each of the plurality of ultrasound sensors of the at least one additional patch and the at least one additional vertebra;
tracking, by the monitoring system, a second orientation and a second location over time of the at least one additional patch relative to the monitoring system; and
displaying on the surgical display a second virtual representation of the second orientation and the second location of the at least one additional vertebra relative to the monitoring system.

17. The surgical tracking method of claim 15, wherein tracking the orientation and the location of the first patch comprises tracking a position over time of a plurality of targets on the first patch using the monitoring system.

18. The surgical tracking method of claim 15, further comprising determining an axial rotation and displacement of the first vertebra relative to the first patch over time based on the measurements of distances between each of the plurality of ultrasound sensors and the first vertebra.

19. The surgical tracking method of claim 15, further comprising, before applying the first patch, imaging the spine of the patient to identify individual vertebrae.

20. The surgical tracking method of claim 15, further comprising, after applying the first patch, imaging the spine of the patient to identify individual vertebrae; and
associating, by the monitoring system, the first patch with the first vertebra in the spine of the patient automatically based on the imaging of the spine and placement of one or more fiducials in the first patch that are imposed on the imaging of the spine.
